(19) United States
(12) Patent Application Publication
(10) Pub. No.: US 2012/0219496 A1
(43) Pub. Date: Aug. 30, 2012

(54) GADOLINIUM-LINKED NANOCLUSTERS

(76) Inventors: Andrew Tsourkas, Philadelphia, PA (US); Zhiliang Cheng, Langhorne, PA (US)

(21) Appl. No.: 13/393,773

(22) PCT Filed: Aug. 30, 2010
(86) PCT No.: PCT/US10/47136

§ 371 Date: May 14, 2012

Related U.S. Application Data
(60) Provisional application No. 61/239,365, filed on Sep. 2, 2009.

(51) Int. Cl. A61K 49/18; C08G 73/00; A61K 51/12; C07F 5/00; B82Y 5/00

(52) U.S. Cl. 424/1.65; 424/9.323; 534/15; 525/451; 428/402; 977/773; 977/930; 977/927

(57) ABSTRACT

The invention relates to nanocluster compositions and uses thereof. Specifically, the invention relates to gadolinium-linked nanocluster compositions and their use in diagnosis and prognosis of diseases. The nanocluster compositions of the invention are effective in enhancing the payload of a gadolinium and thereby increasing the longitudinal relaxivity of each particle in the cluster.

### CROSS-REFERENCE TO RELATED APPLICATIONS

This application claims priority to U.S. provisional patent application 61/239,365, filed Sep. 2, 2009, which is incorporated by reference herein in its entirety.

### FIELD OF THE INVENTION

The invention relates to nanocluster compositions and uses thereof. Specifically, the invention relates to gadolinium-linked nanocluster compositions and their use in diagnosis and prognosis of diseases.

### BACKGROUND OF THE INVENTION

Magnetic resonance (MR) imaging procedures have become common practice in diagnostic clinical medicine owing to their ability to provide high-resolution three-dimensional images of soft tissue. Many of these diagnostic procedures utilize intravenous MR contrast agents, such as gadolinium (Gd), to improve tissue contrast and to provide important information about perfusion, vascular permeability, and extracellular volume. The recent development of targeted paramagnetic contrast agents promises to even further expand the utility of diagnostic MR imaging by providing a mechanism to probe the molecular profile of tissues.

Chelated gadolinium (Gd) is a widely utilized paramagnetic agent that serves as an extracellular fluid contrast agent for magnetic resonance (MR) imaging. Chelated Gd has also been used for imaging intravascular space by conjugating the chelate to proteins and/or polymers that remain within the vasculature. More recently, many attempts have been made to develop Gd-based nanoparticles.

In general, the longitudinal relaxivity (R1) of Gd-based contrast agents is dependent on two key features, the water-exchange rate between bulk water and water bound to the chelated Gd and the rotational correlation time of the Gd ions. The rotational correlation lifetime is generally increased through conjugation of Gd chelates to macromolecular objects. A wide range of macromolecules and other nanoparticulate systems have already been tested as platforms for Gd labeling. Some examples include dendrimers, polymers, liposomes, micelles, emulsions, and silica nanoparticles. For these multimeric gadolinium complexes, it is not only the relaxivity per Gd that defines the effectiveness of the contrast agent but also the number of chelated Gd per nanoparticle. These two parameters can be represented as the relaxivity per nanoparticle. Some of the agents have exhibited relaxivities on the order of 10⁵ to 10⁶ mM⁻¹ s⁻¹ per nanoparticle. Since the R1 for chelated Gd is typically only between 5 and 30 mM⁻¹ s⁻¹ and the theoretical maximum R1 for Gd is estimated to be only ˜100 mM⁻¹ s⁻¹, these contrast agents clearly benefited most from their ability to carry a high Gd payload.

Accordingly, there exits a need for improved nanoplatforms that would provide enhanced R1 per particle.

### SUMMARY OF THE INVENTION

In one embodiment, the invention provides a composition comprising a porous cluster of nanoparticles, each nanoparticle in said cluster linked to a gadolinium ion, wherein said each nanoparticle is further linked to at least one other nanoparticle in said cluster. In an exemplary embodiment, the composition is effective in enhancing the payload of a gadolinium ion.

In another embodiment, the invention provides a contrast agent for magnetic resonance (MR) imaging, comprising a porous cluster of nanoparticles, each nanoparticle in said cluster linked to a gadolinium ion and a targeting ligand, wherein said each nanoparticle is further linked to at least one other nanoparticle in said cluster.

In another embodiment, the invention provides a blood pool agent, comprising a porous cluster of nanoparticles, each nanoparticle in said cluster linked to a gadolinium ion, wherein said each nanoparticle is further linked to at least one other nanoparticle in said cluster.

mom In another embodiment, the invention provides an imaging agent for macrophage infiltration comprising: a porous cluster of nanoparticles, each nanoparticle in said cluster linked to a gadolinium ion and a targeting ligand, wherein said each nanoparticle is further linked to at least one other nanoparticle in said cluster.

In another embodiment, the invention provides an agent for visualizing a cell comprising: a porous cluster of nanoparticles, each nanoparticle in said cluster linked to a gadolinium ion and a targeting ligand, wherein said each nanoparticle is further linked to at least one other nanoparticle in said cluster.

In another embodiment, the invention provides a method for producing a cluster of nanoparticles, comprising the steps of: providing a plurality of nanoparticles; cross-linking each nanoparticle with at least one other nanoparticle; labeling each nanoparticle or said cluster with a chelated gadolinium ion; and functionalizing said cluster with a target agent.

In another embodiment, the invention provides a method of obtaining a magnetic resonance image (MRI), in a subject, comprising administering to said subject a composition comprising a porous cluster of nanoparticles, each nanoparticle in said cluster linked to a gadolinium ion and a targeting ligand, wherein said each nanoparticle is further linked to at least one other nanoparticle in said cluster, whereby the targeting ligand is specific for a marker of a disease in said subject.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood from a reading of the following detailed description taken in conjunction with the drawings in which like reference designators are used to designate like elements, and in which:

FIG. 1A is schematic diagram illustrating approach used to prepare paramagnetic targeted dendrimer nanoclusters (DNCs). Nanoclusters were fabricated by crosslinking polyamidoamine (PAMAM) dendrimers (G5) using a bifunctional amine-reactive crosslinker. Following DNCs formation, paramagnetic Gd³⁺ ions were conjugated into DNCs via DTPA. The resulted paramagnetic NCs were further functionalized with fluorescence imaging probe FITC and tumor-targeted ligand folic acid (FA). FIG. 1B is a standalone schematic of the nanocluster (DNC), according to one embodiment of the invention.

FIG. 2A shows intensity-weighted hydrodynamic diameter of DNCs. The measuring angle of DLS was 90°. FIG. 2B shows a representative transmission electron micrograph (TEM) of DNCs. The image was taken at 80 kV. The particles were observable without additional staining. The scale bar represent 100 nm

FIG. 3 shows relaxivity determination for Gd-DNCs. Measurements were acquired at 1.41 T (60 MHz) at 40° C. Tl measurements were also made for small Gd-DTPA.

FIG. 4 shows cellular uptake of FITC-FA-DNCs. (A&B) untreated KB cells; (C&D) KB cells following 2 hours incubation with FITC-FA-DNCs; (E&F) KB cells following 2 hours incubation with FITC-FA-DNCs in the presence 5 mM folic acid in culture media. A vs. B, C vs. D and E vs. F correspond to KB cells photographed under phase-contrast vs. same cells under fluorescence microscopy.

FIG. 5 shows Tl-weighted magnetic resonance images of KB cell pellets: (A) Untreated KB cells; (B) KB cells incubated with FA-DNCs; (C) KB cells incubated with FA-DNCs in the presence 5 mM folic acid; (D) H₂O.

FIG. 6 shows the viability of KB and NIH 3T3 cells incubated with FA-DNCs. DNCs were incubated with cells at various Gd concentrations for 24 hours. Viability was measured and normalized to cells grown in the absence of any particles based on MTT assay.

FIG. 7 shows magnetic resonance images of nude mice at various time points following the intravenous injection of Gd-contained nanoparticles. The local hyperintensity generated by the Gd-contained nanoparticles was visualized using a 4.7 T small animal MR. The top row shows mice with KB cell tumor xenografts before and after i.v. injection with folate receptor-targeted DNCs. The second row shows mice with KB cell tumor xenografts before and after i.v. injection with folate receptor-targeted DNCs in the presence 50 mM free folic acid (FA). The third row shows mice with KB cell tumor xenografts before and after i.v. injection with folate receptor-targeted, Gd-labeled dendrimers (individual dendrimers, G5). The bottom row shows mice with T6-17 cell tumor xenografts (i.e. folate receptor-negative) before and after i.v. injection with folate receptor-targeted DNCs. Images were acquired pre-injection and 1, 4 and 24 hr post-injection. White arrow shows location of tumor.

FIG. 8 shows a quantitative analysis of the MR images provided in FIG. 7. The average MR signal intensity (SI) was measured for each tumor and the relative signal enhancement, rSE, was then calculated as the quotient of the SI in the 24 hr post-contrast image and the pre-contrast image. A t-test (two-tailed, unequal variance) was used to compare the rSE for each group of animals. A p<0.05 was considered statistically significant and is indicated by an asterisk.

FIG. 9 shows dynamic light scattering histograms of DNCs prepared using NHS-PEG-NHS (blue) and thiol-ene chemistry (pink). Thiol-ene chemistry produces DNCs with significantly less polydispersity.

FIG. 10 shows dynamic light scattering histograms of DNCs with different median hydrodynamic diameters. DNCs were prepared using thiol-ene chemistry.

### DETAILED DESCRIPTION OF THE INVENTION

The invention relates to nanocluster compositions and uses thereof. Specifically, the invention relates to gadolinium-linked nanocluster compositions and their use in diagnosis and prognosis of diseases.

In one embodiment, provided herein is a composition comprising a porous cluster of nanoparticles, each nanoparticle in said cluster linked to a gadolinium ion, wherein said each nanoparticle is further linked to at least one other nanoparticle in said cluster. In an exemplary embodiment, the composition is effective in enhancing the payload of a gadolinium ion. In another embodiment, provided herein is a contrast agent for magnetic resonance (MR) imaging, comprising a porous cluster of nanoparticles, each nanoparticle in said cluster linked to a gadolinium ion and a targeting ligand, wherein said each nanoparticle is further linked to at least one other nanoparticle in said cluster.

In another embodiment, provided herein is a blood pool agent, comprising a porous cluster of nanoparticles, each nanoparticle in said cluster linked to a gadolinium ion, wherein said each nanoparticle is further linked to at least one other nanoparticle in said cluster. In another embodiment, provided herein is an imaging agent for macrophage infiltration comprising: a porous cluster of nanoparticles, each nanoparticle in said cluster linked to a gadolinium ion and a targeting ligand, wherein said each nanoparticle is further linked to at least one other nanoparticle in said cluster. In another embodiment, provided herein is an agent for visualizing a cell comprising: a porous cluster of nanoparticles, each nanoparticle in said cluster linked to a gadolinium ion and a targeting ligand, wherein said each nanoparticle is further linked to at least one other nanoparticle in said cluster.

In another embodiment, provided herein is a method for producing a cluster of nanoparticles, comprising the steps of: providing a plurality of nanoparticles; cross-linking each nanoparticle with at least one other nanoparticle; labeling each nanoparticle with a gadolinium ion; and functionalizing said cluster with a target agent. In another embodiment, provided herein is a method of obtaining a magnetic resonance image (MRI), in a subject, comprising administering to said subject a composition comprising a porous cluster of nanoparticles, each nanoparticle in said cluster linked to a gadolinium ion and a targeting ligand, wherein said each nanoparticle is further linked to at least one other nanoparticle in said cluster, whereby the targeting ligand is specific for a marker of a disease in said subject.

In one embodiment, in contrast to other imaging modalities (i.e. x-ray, nuclear, and ultrasound), the effect of MR contrast agents is not seen directly on the image, but rather it is their effect on proton relaxation, normally water protons, that is observed. In another embodiment, it is the change in relaxation rate of water protons in the presence of a paramagnetic ion, such as lanthanides (e.g. Gd) that is detectable by MR and is responsible for enhancing image contrast. Relaxivity is represented quantitatively as

$$R 1 = \frac{Δ ( \frac{1}{T 1} )}{[ M ]} R 2 = \frac{Δ ( \frac{1}{T 2} )}{[ M ]}$$

where T1 and T2 refer to the relaxation time of protons in the longitudinal and transverse plane respectively and [M] is the concentration of contrast agent or metal ion. Gadolinium is typically used as a T1 agent because they are generally more effective at reducing the longitudinal relaxation rate. Optimal relaxivity of a contrast agents is dependent in one embodiment, on two key features, the water-exchange rate and the rotational correlation lifetime. In one embodiment, faster water exchange rates are preferred, which means that the paramagnetic ion, such as lanthanides (e.g. Gd), should efficiently relax the water that comes in contact with it and the relaxed water should exchange rapidly with the bulk water so that many water molecules can be relaxed by a single paramagnetic ion. Slower rotational correlation lifetimes are also preferred in another embodiment. The rotational correlation lifetime is increased in one embodiment, through conjugation of the paramagnetic ion, such as lanthanides (e.g. Gd) chelate to macromolecular objects.

Accordingly and in one embodiment, provided herein is a composition comprising: a porous nanocluster comprising a plurality of nanoparticles, each nanoparticle in said cluster operably linked to a paramagnetic ion, wherein said each nanocluster is further operably linked to at least one other nanoparticle in said cluster. In one embodiment, the term, “operably linked” refers to the paramagnetic ion or the nanoparticles being arranged so that they function in concert for their intended purposes. In another embodiment, the nanocluster described herein is used in the methods described herein.

As used herein, the term “nanocluster” refers to an association of a plurality of nanoparticles. Preferably, the nanoclusters as described herein comprise approximately 2, 5, 10, 50, 100, 1000, 10,000, 100,000, 300,000, and 500,000 nanoparticles. In other preferred embodiments, the nanoclusters comprise approximately 2-500,000 nanoparticles, approximately 10-500,000 nanoparticles, approximately 1000-400,000 nanoparticles, approximately 50,000-300,000 nanoparticles, or approximately 100,000-300,000 nanoparticles. The size of the nanocluster can depend on the type of nanoparticle used, or other factors known to one of skilled in the art.

As used herein, a “nanoparticle” is defined as a particle having a diameter of from approximately 1 to approximately 500 nanometer (nm), having any size, shape or morphology, known to one of skilled in the art. In one embodiment, the hydrodynamic diameter of each nanoparticle ranges between 1 nm and 500 nm. In another embodiment, the hydrodynamic diameter of each nanoparticle ranges between 50 nm and 300 nm. In another embodiment, the hydrodynamic diameter of each nanoparticle ranges between 100 nm and 200 nm. In one embodiment, the hydrodynamic diameter of each nanoparticle is about 500, 300, 200, 150, 100, 50, or 5 nm. In another embodiment, the hydrodynamic diameter of each nanoparticle is 150 nm.

Examples of a nanoparticle include, but are not limited to, a dendrimer, a polymer, a macromolecule, a peptide, a protein, a polymersome, a multi-functional chelating agent, a nucleic acid, a polylysine, a dextran, or a combination thereof. In one embodiment, the dendrimer is a PAMAM dendrimer. In one embodiment, the dendrimer is a fifth generation PAMAM dendrimer. In another embodiment, the dendrimer is between a second and tenth generation.

Dendritic polymers include, but are not limited to, symmetrical and unsymmetrical branching dendrimers, cascade molecules, arborols, and the like. The PAMAM dendrimers disclosed herein are symmetric, in that the branch arms are of equal length. The branching occurs at the hydrogen atoms of a terminal —NH2 group on a preceding generation branch.

Even though not formed by regular sequential addition of branched layers, hyperbranched polymers, e.g., hyperbranched polyols, may be equivalent to a dendritic polymer where the branching pattern exhibits a degree of regularity approaching that of a dendrimer.

Topological polymers, with size and shape controlled domains, are dendrimers that are associated with each other (as an example covalently bridged or through other association as defined hereafter) through their reactive terminal groups, which are referred to as “bridged dendrimers.” When more than two dense dendrimers are associated together, they are referred to as “aggregates” or “dense star aggregates.” Therefore, dendritic polymers include bridged dendrimers and dendrimer aggregates. Dendritic polymers encompass both generationally monodisperse and generationally polydisperse solutions of dendrimers. The dendrimers in a monodisperse solution are substantially all of the same generation, and hence of uniform size and shape. The dendrimers in a polydisperse solution comprise a distribution of different generation dendrimers.

Dendritic polymers also encompass surface modified dendrimers. For example, the surface of a PAMAM dendrimer may be modified by the addition of an amino acid (e.g., lysine or arginine). As used herein, the term “generation” when referring to a dendrimer means the number of layers of repeating units that are added to the initiator core of the dendrimer. For example, a 1st generation dendrimer comprises an initiator core and one layer of the repeating unit, and a 2nd generation dendrimer comprises an initiator core and two layers of the repeating unit, etc. Sequential building of generations (i.e., generation number and the size and nature of the repeating units) determines the dimensions of the dendrimers and the nature of their interior.

Methods for linking nanoparticles, for example, dendrimers to biological substrates are well known to those of skill in the art, and include the use of a cross-linking agent. For example, thiol-reactive species can be made by coupling the dendrimer hydroxyl group to the isocyanate end of the bi-functional cross-linker, N-(p-maleimidophenyl)isocyanate, leaving a thiol-reactive maleimide for coupling to proteins. In one embodiment, a cross-linking agent is an agent that links one nanoparticle to one or more other nanoparticles; one nanoparticle to a paramagnetic ion (e.g., chelated gadolinium); one paramagnetic ion to another paramagnetic ion; or combinations thereof. Examples of a cross-linking agent include, but are not limited to, a homobifunctional cross-linker, a heterobifunctional cross-linker, a linear polymer, a branched polymer, a nanoparticle, a nucleic acid, a peptide, a protein, or a combination thereof. In a particular embodiment, the cross-linking agent is a homobifunctional amine-reactive cross-linking agent, for example, NHS-PEG-NHS. The presence of PEG spacer arm may help maintain the water solubility of formed dendrimer clusters.

In some embodiments, the cross-linking reaction may be performed by a click-chemistry, preferably, a thiol-ene chemistry. Other suitable click chemistries, known to one of skilled in the art, for example, but not limited to, Staudinger ligation and Cu-catalyzed terminal alkyne-azide cycloaddition, may also be used.

In another embodiment, the paramagnetic ion linked to the nanoparticle described herein is a lanthanide, or a lanthanide-chelated agent in another embodiment, or their combination in yet another discrete embodiment. In another embodiment, the lanthanide used in the methods and porous polymersomes described herein, is Gd³⁺. In another embodiment, the lanthanide used as the paramagnetic ion described and used herein is Eu³⁺, Tm³⁺, Dy³⁺ or Yb³⁺, each a discrete embodiment of the paramagnetic ion used herein.

In another embodiment, the paramagnetic ion is gadolinium (Gd), a chelated gadolinium agent or their combination. The gadolinium chelates currently available for administration worldwide include, but are not limited to, gadopentate dimeglumine (Magnevist from Berlex Laboratories, Wayne, N.J.; and Schering, Berlin Germany), gadodiamide (Omniscan; Nycomed, Princeton, N.J.), gadoteridol (ProHance; Bracco Diagnostic, Princeton, N.J.) and gadoversetamide (OptiMARK; Mallinckrodt, St. Louis, Mo.). Other gadolinium chelates known to one of skilled in the art can also be used.

In one embodiment, the nanocluster of the invention is effective in enhancing the payload of a Gd ion. In another embodiment, the payload ranges between 50 and 500,000. In another embodiment, the payload ranges between 400 and 400,000. In another embodiment, the payload ranges between 100,000 and 10,000,000. In another embodiment, the payload is approximately 10,000,000, 1,000,000, 500,000, 400,000, 300,000, 200,000, 100,000, 50,000, 10,000, 5000, 1000, 500, 100, or 50. In another embodiment, the payload is approximately 300,000. In one embodiment, the porosity of the nanocluster is between about 1 and about 85% (v/v).

“Porosity” refers in one embodiment, to the fractional volume (dimension-less) that is not occupied by solid material. For the nanoparticles described herein, which in another embodiment, are used in the methods provided herein, porosity refers in one embodiment, to the fractional volume of the nanocluster that is not occupied by nanoparticles, paramagnetic ions, chelating agents, and other solid components (e.g., cross-linking agents, targeting ligands). In one embodiment, porosity, as used herein, refers to the void volume that includes the interstitial volume between nanoparticles plus any volume within the nanocluster (i.e., internal porosity)) that is not occupied by the nanoparticle or the paramagnetic lanthanide or the nanoparticle that prevents the paramagnetic ion from leaking out of the nanocluster, or other solid components (e.g., cross-linking agents, targeting ligands).

In one embodiment, the nanocluster of the invention exhibits the longitudinal relaxivity per particle of approximately 3.6×10⁶ mM⁻¹S⁻¹.

In another embodiment, the invention provides a method for producing a cluster of nanoparticles, comprising the steps of: providing a plurality of nanoparticles; cross-linking each nanoparticle with at least one other nanoparticle; labeling each nanoparticle with a gadolinium ion; and functionalizing said cluster with a target agent.

In another embodiment, nanoclusters are produced by crosslinking a source material (e.g., PAMAM dendrimer) in presence of a cross-linking agent known to one of skilled in the art. Uncrosslinked materials can be removed by multiple washes on centrifugal filter devices. Nanoclusters of the invention can also be produced by other methods known to one of skilled in the art. Examples of other methods include, but are not limited to, free radical polymerization and click chemistry.

The purified nanoclusters can be mixed with the ionic (e.g., GdCl3), chelated (e.g., Gd-DTPA), or other forms of gadolinium. The unreacted gadolinium can be removed by multiple washes on centrifugal filter devices. The nanoclusters can be functionalized with a targeting agent. The gadolinium labeled nanoclusters can be linked or coupled to a targeting ligand and/or a fluorescent probe (e.g., fluorescein isothiocyanate) using a method known to one of skilled in the art.

In some embodiments, no functionalization of the nanocluster is needed. For example, the nanoclusters of the invention may be used as a blood pool agent, or to report on macrophage infiltration, or to look at other phagocytic cells. It is known in the art that these applications do not require targeting agents, although for improved cell uptake targeting agents may be used.

In some embodiments, the nanoclusters of the present invention may be used to detect the presence of a particular analyte, for example, a protein, enzyme, polynucleotide, carbohydrate, antibody, or antigen. Molecular analytes include antibodies, antigens, polynucleotides, oligonucleotides, proteins, enzymes, polypeptides, polysaccharides, cofactors, receptors, ligands, and the like. The analyte may be a molecule found directly in a sample such as a body fluid from a host. The sample can be examined directly or may be pretreated to render the analyte more readily detectible. Furthermore, the analyte of interest may be determined by detecting a targeting ligand or agent probative of the analyte of interest such as a specific binding pair member complementary to the analyte of interest, whose presence will be detected only when the analyte of interest is present in a sample. Thus, the targeting ligand or agent probative of the analyte becomes the analyte that is detected in an assay.

In one embodiment, the targeting ligands cover a range of suitable moieties which bind to components of blood clots. In another embodiment, a component may itself be used to generate a ligand by using the component to raise antibodies or to select aptamers that are specific binding partners for the component. In one embodiment, a suitable ligand may be known in the art. In other embodiments, antibodies can be raised to desired components by conventional techniques and can be provided, in certain embodiments, as monoclonal antibodies or fragments thereof, or as single chain antibodies produced recombinantly.

In one embodiment, the targeting ligand is coupled covalently to the nanoparticle. In another embodiment, the targeting ligand used in the methods described herein, is a small molecule, a peptide, a natural binding partner, another protein ligand, an antibody or their combinations. In one embodiment, the targeting ligand is specific for a marker of a pathology of interest.

If in one embodiment, the subject to be administered the compositions described herein is human, it may be desirable to humanize antibody-type ligands using techniques generally known in the art. In other embodiments, suitable proteins which bind to targets can be discovered through phage-display techniques or through the preparation of peptide libraries using other appropriate methods. In one embodiment, selective aptamers which are able selectively to bind desired targets may also be prepared using known techniques such as SELEX™. (Aptamers are oligonucleotides which are selected from random pools for their ability to bind selected targets.) In addition to the foregoing, peptidomimetics, which are small organic molecules intended to mimic peptides of known affinities can also be used as targeting agents. Particularly preferred are targeting agents that bind to fibrin, as fibrin is a particularly characteristic element included in blood clots. Antifibrin antibodies are particularly preferred, including fragments thereof, such as the F_{ab}, F (ab′)₂ fragments, single chain antibodies (Fᵥ) and the like.

In another embodiment, provided herein is a magnetic resonance imaging agent comprising the nanocluster described hereinabove. In one embodiment, the nanocluster described hereinabove, is used in the methods described herein.

In one embodiment, provided herein is a magnetic resonance imaging agent comprising, in another embodiment, a porous nanocluster, comprising the nanoparticles of any one of the embodiments described herein, linked to a ligand, wherein the ligand is specific for a pre-selected marker.

In one embodiment, the nanoclusters described hereinabove, are used in the methods of obtaining a pathology-specific magnetic resonance image (MRI) of a subject.

Accordingly and in one embodiment, provided herein is a method of obtaining a magnetic resonance image (MRI), in a subject, comprising administering to said subject a composition comprising a porous cluster of nanoparticles, each nanoparticle in said cluster linked to a gadolinium ion and a targeting ligand, wherein said each nanoparticle is further linked to at least one other nanoparticle in said cluster, whereby the targeting ligand is specific for a marker of a disease in said subject.

In another embodiment, provided herein is a method of obtaining a pathology-specific magnetic resonance image (MRI) of a subject, comprising administering to said subject a composition comprising a porous cluster of nanoparticles, each nanoparticle in said cluster linked to a gadolinium ion and a targeting ligand, wherein said each nanoparticle is cross-linked to at least one other nanoparticle in said cluster, whereby the targeting ligand is specific for a marker of a disease in said subject.

In one embodiment, the marker is a marker of a cancer, inflammation, autoimmune disease, cardiovascular disease, apoptosis or their combination in other discrete embodiments of the markers used in the methods described herein.

Accordingly, in one embodiment, provided herein is a method of obtaining a breast-cancer specific magnetic resonance image (MRI) of a subject, comprising administering to said subject a nanocluster comprising nanoparticles, wherein at least one of said nanoparticles is linked to an antibody, or a fragment thereof, specific against a carcinoembryonic antigen (CEA), thereby delivering a breast-cancer specific magnetic resonance image (MRI) of a subject.

In another embodiment, the cancer marker for which a specific ligand is linked to the nanoparticles described herein, is a Transferrin receptor, or c-MET, αv-β3 integrins, EGFR, Her2/neu, PSA, a member of the MUC-type mucin family, a member of the epidermal growth factor receptor (EGFR) family, a carcinoembryonic antigen (CEA), a MAGE (melanoma antigen) gene family antigen, a T/Tn antigen, a hormone receptor, a Cluster Designation/Differentiation (CD) antigen, a tumor suppressor gene, a cell cycle regulator, an oncogene, an oncogene receptor, a proliferation marker, an adhesion molecule, a proteinase involved in degradation of extracellular matrix, a malignant transformation related factor, a human carcinoma antigen, a member of the vascular endothelial growth factor (VEGF) receptor family, a glycoprotein antigen, a DF3 antigen, a 4F2 antigen, a MFGM antigen, or their combination in other, discrete embodiments of the cancer markers for which a specific ligand is linked to the nanoparticle described herein. A person skilled in the art, would readilly recognize that many other ligands to many other cancer markers can be used according to the methods described herein and therefore, the list provided is an embodiment and should not limit the broad scope of the ligand/marker combination available, so long as that combination lends itself to being specifically targeted.

In another embodiment, the pathology of interest, sought to be specifically imaged, is cardiovascular disease and the cardiovascular disease marker is VCAM-1, or integrin glcyoprotein IIb/IIIa, myosin, e-selectin, or their combination in other discrete embodiments of the CVD markers for which a specific ligand is linked to the nanocluster described herein.

The paramagnetic metals useful in the MRI contrast agents described herein include rare earth metals, typically, lanthanum, ytterbium, gadolinium, europium, and the like. Iron ions and manganese ions may also be used. Also included in the surface of the nanoparticle, in some embodiments described herein, are biologically active agents. These biologically active agents can be of a wide variety, including proteins, nucleic acids, and the like. Thus, included among suitable pharmaceuticals are antineoplastic agents, hormones, analgesics, anesthetics, neuromuscular blockers, antimicrobials or antiparasitic agents, antiviral agents, interferons, antidiabetics, antihistamines, antitussives, anticoagulants, and the like.

In one embodiment, the inclusion of a chelating agent containing a paramagnetic ion is useful as a magnetic resonance imaging contrast agent. The inclusion of biologically active materials makes them useful as drug delivery systems. The inclusion of radionuclides makes them useful either as therapeutics for radiation treatment or as diagnostics for imaging or both. A multiplicity of such activities may be included; thus, images can be obtained of targeted tissues at the same time active substances are delivered to them.

In another embodiment, a radionuclide may be coupled to the nanoparticle. In a particular embodiment, the radionuclide is ⁹⁹ Tc. Means to attach various radioligands to the nanoparticles described herein are well known in the art.

In one embodiment, the invention provides a composition comprising: a porous cluster of nanoparticles, each nanoparticle in said cluster comprising a multivalent agent that has atleast three functional groups, wherein said each nanoparticle linked to a gadolinium ion, and wherein said each nanoparticle is linked to at least one other nanoparticle in said cluster. Examples of a multivalent agent include, but are not limited to, a polymer, a dendrimer, a polymersome, a macromolecule, a peptide, a protein, a chelating agent, a nucleic acid, a polylysine, a dextran, or a combination thereof.

In one embodiment, the nanoparticle comprises a polymer agent. In one embodiment, the nanoparticle is conjugated to the paramagnetic ion, wherein the nanoparticle is capable of preventing the paramagnetic ion from leaking out of the nanocluster.

In one embodiment, the invention provides a composition comprising: a porous cluster of nanoparticles, each nanoparticle in said cluster comprising a polymersome linked to a gadolinium ion, wherein said each nanoparticle is linked to at least one other nanoparticle in said cluster.

“Polymersomes” refers in certain embodiments to vesicles, which are assembled from synthetic polymers in aqueous solutions. Unlike liposomes, a polymersome does not include lipids or phospholipids as its majority component. Consequently, polymersomes can be thermally, mechanically, and chemically distinct and, in particular, more durable and resilient than the most stable of lipid vesicles. The polymersomes assemble during processes of lamellar swelling, e.g., by film or bulk rehydration or through an additional phoresis step, as described below, or by other known methods. Like liposomes, polymersomes form by “self assembly,” a spontaneous, entropy-driven process of preparing a closed semi-permeable membrane.

In one embodiment, the nanocluster composition of the invention can be combined with one or more other compositions. In one embodiment, the nanocluster composition of the invention can be combined with another composition comprising a liposome. In another embodiment, the nanocluster composition of the invention can be combined with another composition comprising a carbohydrate. In another embodiment, the nanocluster composition of the invention can be combined with another composition comprising a suitable multivalent agent known to one of skilled in the art. In another embodiment, the nanocluster composition of the invention can be combined with another composition comprising a drug. In another embodiment, the nanocluster composition of the invention can be combined with another composition comprising a contrast agent.

In some embodiments, the composition can be formulated in pharmaceutical compositions for in vivo administration, preferably to a mammal, more preferably to a human. These compositions can comprise, in addition to one or more of the compounds of the invention, a pharmaceutically acceptable excipient, carrier, buffer, stabiliser or other materials well known to those skilled in the art. Such materials are preferably non-toxic and may not interfere with the function of the components in the composition. The precise nature of the carrier or other material can depend on the route of administration, e.g. intravenous, cutaneous or subcutaneous, nasal, intramuscular, intraperitoneal routes.

The mode of administration, the dosage and frequency of dosage is governed by the mode of administration and dosage considerations conventionally employed with the contrast agent. Typically, these agents are administered by intravenous injection immediately prior to subjecting the patient to a magnetic resonance imaging procedure. Other routes of administration may be utilized as dictated by medical and pharmacological practice related to the desired use of the particular contrast agent employed.

It will be understood that the specific dose level for any particular patient will depend upon a variety of factors including the specific agent employed, the age, body weight, general health, sex, diet, time of administration, route of administration and rate of excretion. Examples of the techniques and protocols mentioned above can be found in Remington's Pharmaceutical Sciences, 16th edition, Osol, A. (ed), 1980.

The term “subject,” as used herein, includes any human or non-human animal. The term “non-human animal” includes all vertebrates, e.g., mammals and non-mammals, such as non-human primates, sheep, dog, cow, chickens, amphibians, reptiles, etc.

The following examples are presented in order to more fully illustrate the preferred embodiments of the invention. They should in no way be construed, however, as limiting the broad scope of the invention.

### EXAMPLES

### Example 1

### Experimental Details

Materials. PAMAM dendrimers (ethylenediamine core, generation 5) were purchased as methanol solutions from Dendritech Inc. (Midland, Mich.). Prior to use, the methanol was removed using nitrogen stream at room temperature. Homobifunctional amine-reactive crosslinker with polyethylene glycol (PEG) spacer arms BS(PEG)₅ (Cat 21581, MW 532.50) was purchased from Pierce. Fluorescein isothiocyanate (FITC) was obtained from Molecular Probes. Diethylenetriaminepentaacetic acid dianhydride (DTPA dianhydride), folic acid and gadolinium (III) chloride were obtained from Sigma-Aldrich. All buffer solutions were prepared with ultrapure grade water.

Synthesis of PAMAM nanocluster. 300 mg PAMAM was dissolved in 30 mL sodium bicarbonate buffer (0.1 M, pH 9.5) and crosslinked by adding 54 μL BS(PEG)₅ (250 mM in DMSO) for 20 hours. Uncrosslinked dendrimers were removed by multiple washes on centrifugal filter devices (Amicon ultrafree-CL, 0.1 μm, Millipore Corp.). 1 g DTPA dianhydride was added to the crosslinked dendrimer sample and maintained at pH 9.5 with NaOH over the reaction time of 10 h. The unreacted DTPA dianhydride was removed by multiple washes on centrifugal filter devices (Amicon Ultra-4, 100K MWCO, Millipore Corp.). The purified DTPA conjugated dendrimer nanoclusters were mixed with 600 mg GdCl₃ in 0.1 M citrate buffer (pH 5.6) overnight at 42° C. The unreacted Gd³⁺ was removed by multiple washes on centrifugal filter devices (Amicon Ultra-4, 5000 MWCO, Millipore Corp.). To ensure complete removal of unreacted Gd³⁺, the Tl relaxation time of the eluent was checked after each centrifugation until no Gd³⁺ was detectable, i.e. until the Tl-relaxation time was equivalent to that of sodium phosphate (pH 7.0) buffer (˜1000 ms).

Folic acid and FITC conjugation. Folic acid was conjugated to dendrimer nanocluster through its surface residue amine groups. For this reaction, the active ester of folic acid was prepared as described previously. 50 μL NHS-folate (10 mg/mL in DMSO) was mixed with 2 mL Gd-DNCs in sodium bicarbonate buffer (0.1 M, pH 9.5). After 2 hours reaction, another 10 μL FITC (5 mg/mL in DMSO) was added and allowed another 30 minutes hour reaction. The unreacted NHS-folate and FITC were removed using a PD-10 column size exclusion column (Amersham Biosciences).

Viscosity Measurements. The viscosity of the dendrimer nanoclusters-solution was obtained using a Cannon-manning semi-micro capillary viscometer (Cannon Instrument CO, State College, Pa.). Deionized water was used to prepare all the samples.

Cell culture. Both KB cells (human nasopharyngeal epidermoid carcinoma cells) and NIH 3T3 cells (mouse fibroblast cells) were purchased from the America Type Tissue Collection (ATCC). KB cells were cultured in RPMI 1640 folic acid free media with 10% fetal bovine serum (FBS). NIH 3T3 cells were cultured in Dulbecco's Modified Eagle's medium with 10% FBS. Both cells were grown at 37° C. in a humidified atmosphere containing 5% CO2.

Cell pellets. KB cells were seeded in four 75 cm² cell-culture flasks at a density of 20×10⁶ cells/flask and cultured overnight. The first flask was incubated with FA-DNCs at Gd concentration 5 mM for 2 hours. The second flask was treated same as the first flask but in the presence of 5 mM free folic acid in the media. The third flask was incubated with pure media for 2 hours. For cell harvesting, KB cells were detached using enzyme free cell dissociation solution and transferred into three falcon tubes. After centrifugation, the supernatant was discarded and the cells were washed with PBS three times. The cells were finally pelleted in PCR tubes.

Cell viability via MTT assay. KB or NIH 3T3 cells were seeded in 96-well plates at a density of 10,000 cells per well. After incubation overnight (37° C., 5% CO2), the medium in each well was aspirated off and loaded with 100 μL of fresh medium containing nanoclusters with different Gd³⁺ concentration. After 24 hours incubation, the medium containing nanoparticles in each well was aspirated off and replaced with 100 μL of medium and 10 μL of MTT reagent. The cells were incubated for 2 to 4 hours, then 100 μL detergent reagent was added and left at room temperature in the dark for 2 hours. The absorbance at 570 nm was measured using a microplate reader (BioTek Instruments, Inc).

Tumor Implantation. Adult female nude mice (average 20 g) were obtained from Harlan Laboratories at 8-weeks of age and fed AIN-93G (TestDiet, Richmond, Ind.). All experiments conformed to animal care protocols approved by the Institutional Animal Care and Use Committee (IACUC) of the University of Pennsylvania. To induce a tumor, ˜2×10⁶ KB cells were suspended into 200 μL of PBS and injected into the right lower back of lightly anesthetized mice (how much isoflurane).

Administration of DNCs. At 9 days after tumor implantation, the tumor size was around 5 mm, and pre-contrast MR images were obtained as described below. Immediately afterwards, the first group (n=3) was treated with FA-DNCs. The second group (n=3) was treated with FA-DNCs and free folic acid. The third group (n=3) was treated with FA-G5 (G5: individual uncrosslinked Gd-labeled PAMAM dendrimer). In all cases, 200 μL sample in PBS were injected into mice through I.V. at the Gd dose of 0.3 mmol/kg. For the second group, the free folic acid concentration was 50 mM.

MR Imaging and Image Processing. Imaging was performed using a 4.7 T small animal horizontal bore Varian INOVA system. Gradients used in the magnet were 12 cm diameter at 25 G/cm. Imaging was performed using a custom-built 50 mm diameter send-receive birdcage volume coil. Following induction of a mouse to the plane of anesthesia using 4% isoflurane/oxygen, mice were fixed to an acrylic patient bed in the prone position and maintained on a 1% isoflurane/oxygen mixture. Body temperature was monitored with a platinum rectal probe connected to a small-animal monitoring system (SA Incorporated) and maintained using a stream of heated air. Tl-weighted images were acquired in the coronal plane using a spin-echo sequence with the following parameters; TR/TE=500/15 milliseconds, matrix=128×128, FOV=30×30 mm, slice thickness=1 mm, NEX=4). Scans were completed prior to, 1, 4 and 24 hours after injection nanoparticles in different conditions, as described above.

For quantification of signal enhancement by MRI contrast agents, signal enhancement ratio R was determined from mean tumor signal intensity values at post and pre contrast agent injection according to the following equation:

R =It24/ItO

where It24 is the mean signal intensity of the tumor 24 hours post injection contrast, ItO is the signal intensity of the tumor pre injection contrast agent.

Instrumentation. Dynamic light scattering (DLS) measurements were performed on a Zetasizer Nano from Malvern Instruments. The scattering angle was held constant at 90°. Microscopy measurements were performed on an Olympus IX 81 motorized inverted fluorescence microscope equipped with an Andor DU897 EMCCD, an X-Cite 120 excitation source (EXFO) and Sutter excitation and emission filter wheels. Image of FITC was acquired using the filter set HQ480/40, HQ535/50, Q505LP. Filter set was purchased from Chroma. Tl-relaxation times were determined using a Bruker mq60 MR relaxometer operating at 1.41 T (60 MHz). ICP-AES was performed by VHG Laboratories (Manchester, N.H.). Transmission electron microscopy (TEM) was performed with a JEOL 2010 electron microscope operated at 80 kV.

### Example 2

### Gd-Conjugated Dendrimer Nanoclusters as a Tumor-Targeted T1 Magnetic Resonance Imagining Contrast Agent

Considering that most current nanoplatforms are only labeled with Gd chelates on their outer surface, to ensure high water accessibility, the inventors of the instant application have found that higher Gd payloads could simply be achieved through the development of highly porous nanoparticles that contained a high Gd content throughout the intraparticular volume. The inventors have shown that this could be accomplished by creating “dendrimer nanoclusters” (DNCs) composed of individual Gd-labeled PAMAM dendrimers that have been cross-linked to form larger nanoparticulate carriers.

Chemically cross-linked dendrimer nanoclusters (DNCs), 150 nm in diameter, have been developed as a platform for preparing targeted magnetic resonance (MR) contrast agents. The large surface area of the porous DNCs allowed for the conjugation of ˜300,000 Gd-DTPA per particle. As a result, the DNCs exhibited an R1 relaxivity of 12.3 mM⁻¹ s⁻¹/Gd and 3.6×10⁶ mM⁻¹ s⁻¹/particle. The DNCs were also labeled with the fluorescent dye, fluorescein isothiocyanate (FITC), and the tumor specific targeting agent, folic acid. The DNCs were capable of specifically binding folate receptor-positive tumor cells both in vitro and in a tumor xenograft mouse model. Due to the extremely high Gd payload, the DNCs exhibited a significant improvement in tumor contrast compared with pre-contrast images and folate-targeted Gd-labeled dendrimers. Therefore, it is determined that these targeted DNCs can potentially serve as an ultrasensitive MR contrast agent for the specific detection of cancer and other disease pathologies.

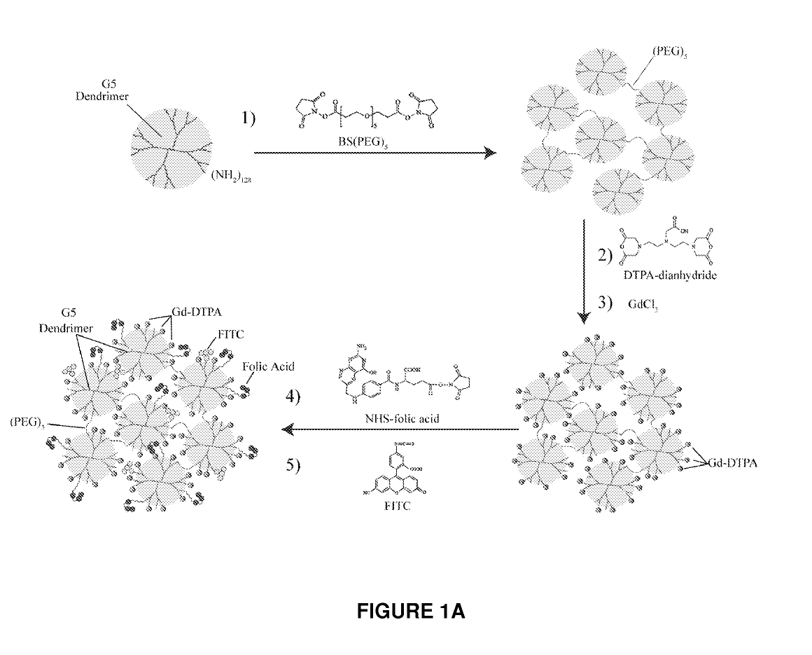

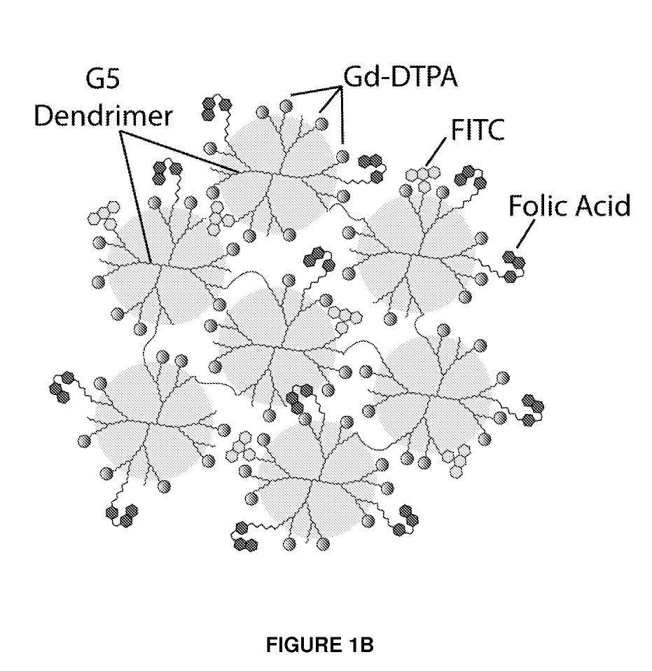

The inventors of the instant application have demonstrated that the Gd-labeled DNCs can readily be functionalized with targeting ligands (e.g. folic acid) and used for in vivo molecular imaging. A schematic of a folate-receptor targeted Gd-labeled DNC is shown in FIG. 1.

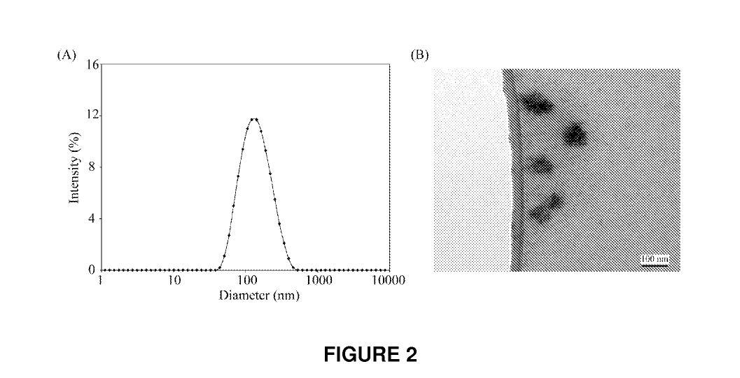

Paramagnetic DNCs were prepared by first crosslinking PAMAM dendrimers (Generation 5) with the homobifunctional amine-reactive crosslinking agent, NHS-PEG-NHS. The presence of the polyethylene glycol (PEG) spacer arm helped maintain the high water solubility of-the formed dendrimer clusters. To control nanocluster size, the molar ratio between NH₂-containing PAMAM dendrimer and NHS-containing BS(PEG)s cross-linker was varied. It was found that at a molar ratio of 50:1 [NH2]:[NHS], it was possible to obtain DNCs with an average hydrodynamic diameter of 150 nm and a relatively narrow size distribution, as determined by dynamic light scattering (DLS) (FIG. 2A). It should be mentioned that non-crosslinked individual dendrimers, with an average diameter of 5.8 nm, were removed through repeated washes on a 100 nm centrifugal filter device. The purified DNCs were labeled with Gd by reacting the surface amines with the chelating agent diethylenetriaminopentaacetic acid (DTPA)-dianhydride. The resulting paramagnetic DNCs were further functionalized with the optical imaging dye fluorescein isothiocyanate (FITC) and the tumor-targeting ligand folic acid.

Transmission electron microscopy (TEM) confirmed the labeling of the DNCs with Gd (FIG. 2B). Due to the presence of the electron-dense gadolinium ions, DNCs were directly placed on a carbon-coated copper grid and observed without using any additional staining agents, which is often required to enhance the contrast of unmodified dendrimers. The DNCs observed by TEM were approximately spherical in shape and 75-150 nm in diameter.

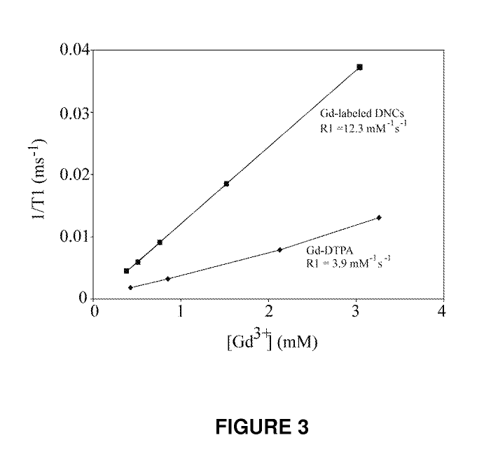

To assess the paramagnetic properties of the Gd-conjugated DNCs, the amount of Gd within the sample was determined by inductively coupled plasma atomic emission spectroscopy (ICP-AES). The relaxivity was then calculated as the slope of the curves 1/Tl vs. Gd concentration, as shown in FIG. 3. Tl-relaxation times were determined using a Bruker mq60 MR relaxometer operating at 1.41 T (60 MHz) and at 40° C. It was found that Gd-conjugated DNCs had an R1 relaxivity value of 12.3 mM⁻¹ s⁻¹ per Gd. This was only slightly higher that Gd-labeled individual PAMAM (G5) dendrimers, which had an R1 relaxivity of 10.1 mM⁻¹ s⁻¹ per Gd. It is likely that only a marginal increase in R1 was observed, despite the larger size of DNCs, due to the “saturation” of ion relaxivity, which has also previously been reported for high generation dendrimers. As a point of comparison, Gd-DTPA was determined to have an R1 of 3.9 mM⁻¹ s⁻¹ per Gd.

In order to determine the R1 per DNC particle, it was first necessary to determine the number of particles within a given aqueous sample. This was accomplished by using Einstein's viscosity equation to determine the DNC volume fraction within various samples and DLS measurements to determine the average volume of individual DNCs. Subsequent measurements of Gd content in the same samples by ICP-AES revealed that there were approximately 300,000 Gd per DNC. For comparison, individual G5 dendrimers possess a maximum of 128 functional groups for attachment of single Gd chelates. The paucity of conjugation sites obviously limits the Gd payload of the particle. Higher generation dendrimers can be used to carry higher payloads, but these dendrimers are difficult to synthesize and costly. Further, even a generation 10 dendrimer can only accommodate a maximum of 4096 single Gd chelates. The dramatic difference in Gd payload between DNCs and individual dendrimers arises from both the larger size of the DNCs and the ability of DNCs to carry chelated Gd throughout the intraparticular volume, not just on its outer shell. Based on the average Gd content of each DNC and the relaxivity per Gd, it is estimated that the relaxivity per DNC is approximately 3.6×10⁶ mM⁻¹ s⁻¹.

The paramagnetic properties of the DNCs reported here compare very favorably with Gd-based agents that have previously been reported in the literature. For example, Gd-labeled shell-crosslinked nanoparticles (40 nm diameter) exhibit an R1 of 39 mM⁻¹ s⁻¹ per Gd (0.47 T) but possess only 510 Gd per particle, which results in an R1 of 2×10⁴ mM⁻¹ s⁻¹ per nanoparticle. Paramagnetic silica nanoparticles (˜100 nm) have been found to exhibit an R1 of 9.0 mM⁻¹ s⁻¹ per Gd (4.7 T) and contain 16,000 Gd per nanoparticle, which results in an R1 of 1.4×10⁵ mM⁻¹ s⁻¹ per nanoparticle. Gd-encapsulated porous polymersomes (˜125 nm) possess nearly 44,000 Gd per particle and exhibit an R1 of 3.2×10⁵ mM⁻¹ s⁻¹ per nanoparticle. Consequently, all three particles exhibit relaxivities that are significantly lower than the paramagnetic DNCs presented here.

Perfluorocarbon nanoparticles have a reported R1 of 25.3 mM⁻¹ s⁻¹ per Gd (1.5 T) and 94,200 Gd per particle,(REF) which results in an R1 of 2.38×10⁶ mM⁻¹ s⁻¹ per nanoparticle; however, while this relaxivity is similar to that of the 150 nm DNCs, it should be noted that the perfluorocarbon particles are much larger with a diameter of 273 nm. Scaling with volume, a paramagnetic DNC of this size would possess 1,800,000 Gd and exhibit an R1 of 2.2×10⁷.

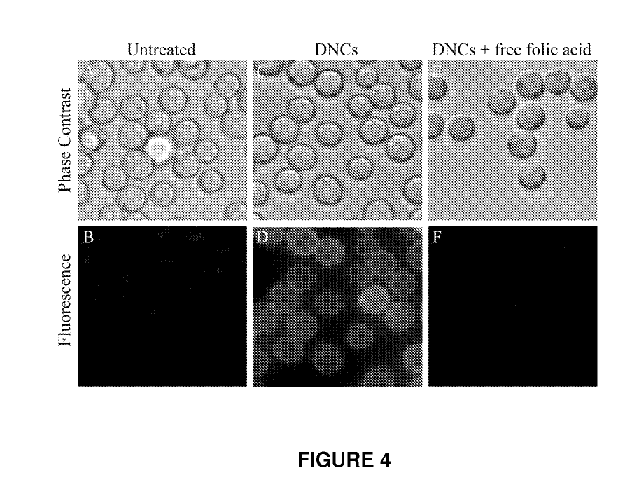

To confirm the folate receptor-targeting capabilities of the DNCs, KB cells were incubated with DNCs for two hours and subsequently analyzed by fluorescence microscopy. It is noted that all of the DNCs were labeled with FITC, in addition to the folic acid and Gd-DTPA. Indicative of cell labeling, all of the KB cells exhibited a bright fluorescent signal (FIG. 4D). To verify that uptake of the DNCs was mediated through folate-receptor-dependent targeting, competitive inhibition studies were conducted by adding DNCs to cell cultures in the presence of excess free folic acid. Under these conditions, fluorescence was significantly reduced (FIG. 4F), confirming that cellular binding of DNCs was specifically mediated by the folate receptor.

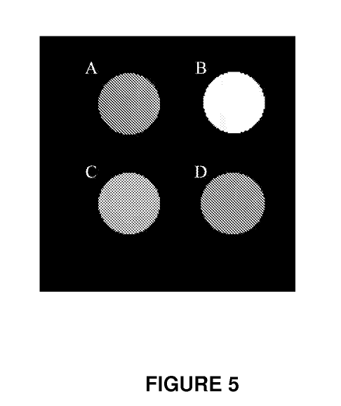

Cell labeling with DNCs was further assessed by acquiring Tl-weighted magnetic resonance (MR) images of KB cells that were pelleted in PCR tubes, following incubation with DNCs in the presence and absence of free folic acid (FIG. 5). It was found that KB cells that were incubated with DNCs alone exhibited a significant enhancement in MR signal intensity, compared with unlabeled cells. Conversely, cells that had been incubated with DNCs in the presence of excess free folic acid only exhibited a slight increase in signal intensity, indicating that the free folic acid was able to specifically block the binding of the DNCs. These results further confirm that DNCs can efficiently bind KB cells via the folate receptor.

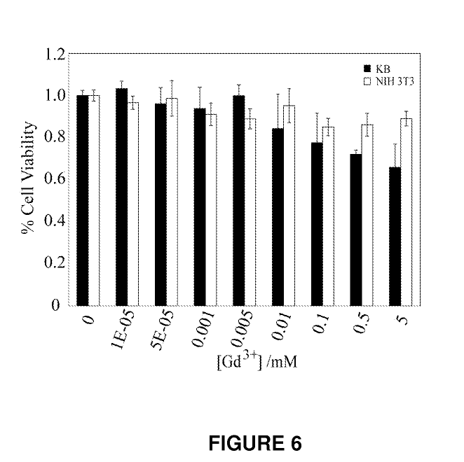

Prior to evaluating DNCs in living subjects, their cytotoxic effects were examined in a MTT cell proliferation assay (where MTT is 3-(4,5-dimethylthiazol-2-yl)2,5-diphenyl-tetrazoilum bromide). Specifically, various concentrations of the folate receptor-targeted DNCs were incubated with KB cells, which are known to express the folate receptor, and NTH 3T3 cells which do not, for 24 hours. The data shown in FIG. 6 indicate the cell viabilities for each cell type normalized to a control cell sample that was not incubated with any DNCs. In general, DNCs did not seem to have much of an effect on the viability of NIH 3T3 cells up to Gd concentration 5 mM (i.e. 90% viability). However, Gd concentrations of 0.1 mM and above did seem to have a significant effect on the viability of KB cells. The high driving force for cell internalization imparted by the folic acid on the DNCs is likely attributable to the KB cell death.

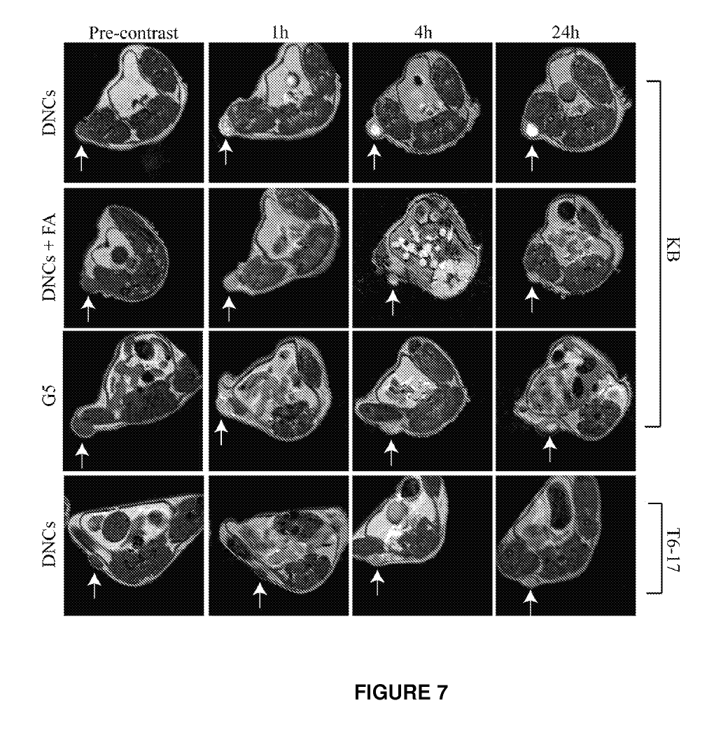

To examine whether DNCs could be used to effectively identify folate-positive tumors in living subjects, axial MR images of mice with subcutaneous KB cell xenografts were acquired precontrast and at various times after intravenous (i.v) injection of DNCs (0.3 mmol Gd/kg) (FIG. 7). In the precontrast images, there was little intrinsic contrast between the implanted tumors and surrounding muscle. At 1 hour following administration of the DNCs, a slight contrast enhancement was observed within the tumor. The signal enhancement increased significantly by 4 hours and by 24 hours the signal within the tumor was extremely bright and the boundary of the tumor was clearly demarcated.

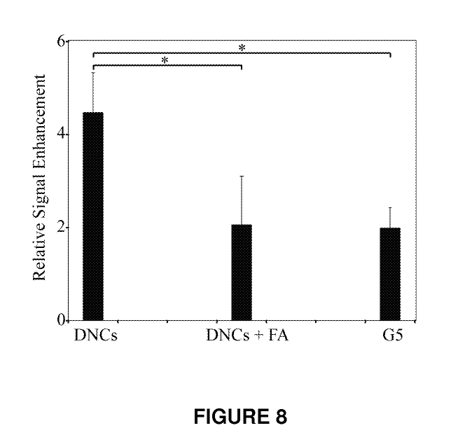

Control experiments to assess specificity were performed by i.v. injection of DNCs in the presence of free folic acid (50 mM). In these animals, a slight enhancement in signal within the tumor was observed; however, the signal was clearly lower than when DNCs were administered alone for each of the time points studied. As an additional control, the signal enhancment of DNCs was compared to Gd-labeled G5 dendrimers, which were also functionalized with folic acid. The targeted dendrimers did exhibit a slight enhancement in signal within the tumor, compared the precontrast images; however, the signal was noticeably lower than that observed with DNCs. Quantitative analysis of the MR images is presented in FIG. 8. These results confirm that the presence of free folic acid led a statistically significant reduction in DNC binding to tumor cells (p<0 05), confirming the specificity of the folate-receptor targeting. The residual signal that was observed even in the presence of folic acid (i.e. Post/Pre>1) is suspected to be due to incomplete blocking of the folate receptor (especially considering the rapid clearance of free folic acid) and because of the enhanced permeability and retention effect within the tumor. Analysis of the MR images also revealed that DNCs exhibited a statistically significant improvement in image contrast compared with targeted dendrimers (p<0.05).

In conclusion, the inventors of the instant application have provided a facile method for the synthesis of nanometer-sized dendrimer nanoclusters that possess a high capacity for Gd-DTPA labeling. Further, the inventors have demonstrated the utility of these DNCs as optical and MR imaging contrast agents for the in vitro and in vivo detection of tumor cells overexpressing the folate receptor. By conjugating appropriate cancer-targeting ligands, various types of cancers can be detected by the ultrasensitive MR. Therefore, the DNCs described here provide a powerful new platform for the early detection of disease.

### Example 3

### Thiol-Ene Chemistry Allows for Improved Control Over DNC Size and Polydispersity

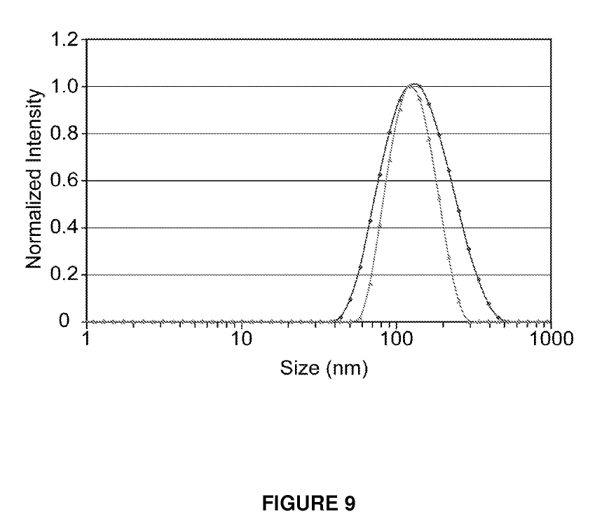

Several methods can be used to reduce polydispersity. The inventors of the instant application have established a facile method for the preparation of DNCs with tunable median sizes and with reduced polydispersity. Specifically, DNCs have been prepared by first reacting PAMAM dendrimers with NHS-acrylate (TCI America). Unreacted NHS-acrylate was removed by performing several ether washes. The dendrimers were then cross-linked in a very controlled fashion by the addition of dithiol PEG linkers. This chemistry is often referred to as thiol-ene chemistry and is considered to be a highly efficient ‘click’ reaction. The improvement in polydispersity that was achieved with thiol-ene chemistry, compared with our previous formulations, which used NHS-PEG-NHS as a crosslinker, is shown in FIG. 9. Further improvement in polydispersity can be made by fine-tuning this protocol. Other approaches to further improve polydispersity include filtration through appropriately sized filters, FPLC, and DNC synthesis within water-in-oil emulsions.

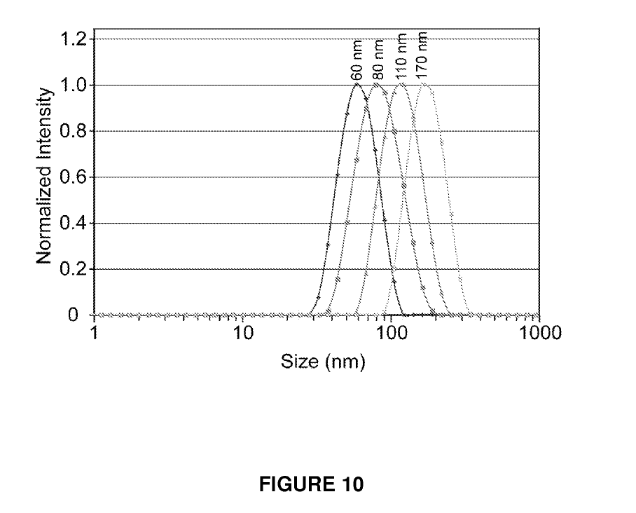

To date, dithiol linkers of various lengths (˜250 Da to ˜800 Da) have already been tested and similar DNC size distributions have been achieved for each. Longer linkages (i.e. up to 2 kDa or longer) may also be tested and they may have effect on Gd relaxivity. In addition to controlling linker length, it was found that the median DNC size can be controlled by adjusting the molar ratios of dendrimer, acrylate, and dithiols. Dynamic light scattering histograms for DNCs of various sizes are shown in FIG. 10. A broader range of DNC sizes can also be made.

Aside from the high reaction efficiency of thiol-ene chemistry, another unique advantage of this approach is that any dithiol species can be used to cross-link PAMAM dendrimers. Therefore, similar results may be accomplished when utilizing oligonucleotides and peptides (with two thiols) to cross-link dendrimers into higher order DNCs. Further, the inventors have also shown that DNCs can be prepared by performing the inverse reaction, where PAMAM dendrimers are labeled with thiols and subsequently cross-linked upon the addition of diacrylates. Therefore, there is strong evidence that this chemistry is highly versatile, repeatable, and highly efficient. Other click chemistries (Staudinger ligation, Cu-catalyzed terminal alkyne-azide cycloaddition, etc.) may also be used.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A composition comprising a porous cluster of nanoparticles, each nanoparticle in said cluster linked to a gadolinium ion, wherein said each nanoparticle is further linked to at least one other nanoparticle in said cluster.

2. The composition of claim 1, wherein said composition is effective in enhancing the payload of a gadolinium ion.

3. The composition of claim 1, wherein said cluster exhibits the longitudinal relaxivity of more than 100 mM⁻¹S⁻¹ per particle.

4. The composition of claim 1, wherein the hydrodynamic diameter of each nanoparticle is about 150 nm.

5. The composition of claim 1, wherein at least one nanoparticle of said cluster is linked to a ligand, wherein the ligand is specific for a pre-selected marker.

6. The composition of claim 1, wherein said nanoparticle is a polymer.

7. The composition of claim 1, wherein said nanoparticle is a dendrimer.

8. The composition of claim 7, wherein said dendrimer is a PAMAM dendrimer.

9. The composition of claim 1, wherein said nanoparticle is a polymersome.

10. The composition of claim 1, wherein said nanoparticle is a polymer, a dendrimer, a polymersome, a liposome, a macromolecule, a peptide, a protein, a chelating agent, a nucleic acid, a polylysine, a dextran, or a combination thereof.

11. The composition of claim 1, wherein the porosity of said cluster is between about 1 and about 85% (v/v).

12. The composition of claim 1, comprising no less than about 50 gadolinium ions.

13. The composition of claim 1, wherein said nanoparticle is conjugated to said gadolinium ion.

14. The composition of claim 1, wherein said composition comprises a cross-linking agent that links said each nanoparticle to said atleast one other nanoparticle.

15. The composition of claim 1, wherein said composition comprises a cross-linking agent that links said each nanoparticle to said chelated gadolinium ion; said chelated gadolinium ion to another chelated gadolinium ion; or combinations thereof.

16. The composition of claim 14 or 15, wherein said cross-linking agent is a homobifunctional crosslinker, a heterobifunctional cross-linker, a linear polymer, a branched polymer, a nanoparticle, a nucleic acid, a peptide, a protein, or a combination thereof.

17. The composition of claim 14 or 15, wherein said cross-linking agent is NHS-PEG-NHS.

18. The composition of claim 14 or 15, wherein said cross-linking agent is a thiol-ene chemistry linker.

19. A magnetic resonance imaging agent comprising the composition of claim 1.

20. A blood pool agent comprising the composition of claim 1.

21. An imaging agent for macrophage infiltration comprising the composition of claim 1.

22. A method for producing a cluster of nanoparticles, comprising the steps of:
providing a plurality of nanoparticles;
cross-linking each nanoparticle with at least one other nanoparticle;
labeling each nanoparticle with a gadolinium ion; and
functionalizing said cluster with a target agent.

23. The method of claim 22, the step of cross-linking is performed in presence of a cross-linking agent.

24. The method of claim 23, wherein the molar ratio of said nanoparticle and said cross-linking agent is approximately 50:1.

25. The method of claim 22, wherein the cross-linked cluster of nanoparticles is effective in enhancing the payload of a gadolinium ion.

26. The method of claim 22, wherein said cluster exhibits R1 relaxivity of more than 100 mM⁻¹S⁻¹ per particle.

27. The method of claim 22, wherein the hydrodynamic diameter of each nanoparticle of said cluster is about 150 nm.

28. The method of claim 22, wherein at least one nanoparticle of said cluster is linked to a ligand, wherein the ligand is specific for a pre-selected marker.

29. The method of claim 22, wherein said nanoparticle is a polymer.

30. The method of claim 22, wherein said nanoparticle is a dendrimer.

31. The method of claim 30, wherein said dendrimer is a PAMAM dendrimer.

32. The method of claim 22, wherein said nanoparticle is a polymersome.

33. The method of claim 22, wherein said nanoparticle is a polymer, a dendrimer, a polymersome, a liposome, a macromolecule, a peptide, a protein, a chelating agent, a nucleic acid, a polylysine, a dextran, or a combination thereof.

34. The method of claim 22, wherein the porosity of said cluster is between about 1 and about 85% (v/v).

35. The method of claim 22, wherein said cluster comprises no less than about 50 gadolinium ions.

36. The method of claim 22, wherein said nanoparticle is conjugated to said gadolinium ion.

37. A method of obtaining a magnetic resonance image (MRI), in a subject, comprising administering to said subject a composition comprising a porous cluster of nanoparticles, each nanoparticle in said cluster linked to a gadolinium ion and a targeting ligand, wherein said each nanoparticle is further linked to at least one other nanoparticle in said cluster, whereby the targeting ligand is specific for a marker of a disease in said subject.

38. The method of claim 37, whereby the targeting ligand is coupled covalently to said nanoparticle.

39. The method of claim 37, whereby the targeting ligand is a small molecule, a peptide, a natural binding partner, another protein ligand, an antibody or their combination.

40. The method of claim 37, whereby the marker is a marker of a cancer, inflammation, an autoimmune disease, a cardiovascular disease, apoptosis or their combination.

41. The method of claim 40, whereby the cancer marker is a Transferrin receptor, c-MET, αv-β3 integrins, EGFR, Her2/neu, PSA, a member of the MUC-type mucin family, a member of the epidermal growth factor receptor (EGFR) family, a carcinoembryonic antigen (CEA), a MAGE (melanoma antigen) gene family antigen, a T/Tn antigen, a hormone receptor, a Cluster Designation/Differentiation (CD) antigen, a tumor suppressor gene, a cell cycle regulator, an oncogene, an oncogene receptor, a proliferation marker, an adhesion molecule, a proteinase involved in degradation of extracellular matrix, a malignant transformation related factor, a human carcinoma antigen, a member of the vascular endothelial growth factor (VEGF) receptor family, a glycoprotein antigen, a DF3 antigen, a 4F2 antigen, a MFGM antigen, or their combination.

42. The method of claim 40, whereby the cardiovascular disease marker is VCAM-1, integrin glcyoprotein IIb/IIIa, myosin, e-selectin, or their combination.

43. The method of claim 40, whereby the apoptosis marker is synaptotagmin I, phophatidylserine or their combination.

44. A contrast agent for magnetic resonance (MR), computerized tomography (CT), or X-ray imaging, comprising a porous cluster of nanoparticles, each nanoparticle in said cluster linked to a gadolinium ion and a targeting ligand, wherein said each nanoparticle is further linked to at least one other nanoparticle in said cluster.

45. A blood pool agent, comprising a porous cluster of nanoparticles, each nanoparticle in said cluster linked to a gadolinium ion, wherein said each nanoparticle is further linked to at least one other nanoparticle in said cluster.

46. An imaging agent for macrophage infiltration comprising: a porous cluster of nanoparticles, each nanoparticle in said cluster linked to a gadolinium ion and a targeting ligand, wherein said each nanoparticle is further linked to at least one other nanoparticle in said cluster.

47. An agent for visualizing a cell comprising: a porous cluster of nanoparticles, each nanoparticle in said cluster linked to a gadolinium ion and a targeting ligand, wherein said each nanoparticle is further linked to at least one other nanoparticle in said cluster.

48. The composition of claim 1, further comprising a drug.

49. The composition of claim 1, further comprising a radionuclide coupled to said cluster.
